(19) United States
(12) Patent Application Publication
(10) Pub. No.: US 2024/0054649 A1
(43) Pub. Date: Feb. 15, 2024

(54) NONINVASIVE HEMOGLOBIN DETECTOR

(71) Applicant: Introduction to Affiliated Hospital of Zunyi Medical University, Zunyi (CN)

(72) Inventors: Xuheng JIANG, Zunyi (CN); Jian YANG, Zunyi (CN); Yu NI, Zunyi (CN); Anyong YU, Zunyi (CN); Mo LI, Zunyi (CN); Ji ZHANG, Zunyi (CN); Xiaofei HUANG, Zunyi (CN); Quan HU, Zunyi (CN); Shuhong WANG, Zunyi (CN); Jianghua CHEN, Zunyi (CN)

(21) Appl. No.: 18/356,687

(22) Filed: Jul. 21, 2023

(30) Foreign Application Priority Data
Aug. 12, 2022 (CN) 202210971394.X

(51) Int. Cl. G06T 7/00; A61B 5/00; H04N 1/00; H04N 23/63

(52) U.S. Cl. CPC G06T 7/0014; A61B 5/0077; H04N 1/00278; H04N 23/631; A61B 2560/0223; G06T 2207/20224; G06T 2207/30041

(57) ABSTRACT

Provided is a noninvasive hemoglobin detector, including an image acquisition system, an image processing and analysis system and a printing device. The image acquisition system is connected with the image processing and analysis system, and the image acquisition system is used for acquiring, transmitting and storing an image to be tested. The image processing and analysis system is further connected with the printing device, and the image processing and analysis system is used for processing and analyzing the image to be tested to obtain an analysis result. The printing device is used for printing the image to be tested and the analysis result.

### CROSS-REFERENCE TO RELATED APPLICATIONS

This disclosure claims priority to Chinese Patent Application No. 202210971394.X, filed on Aug. 12, 2022, the contents of which are hereby incorporated by reference.

### TECHNICAL FIELD

The disclosure belongs to the technical field of detectors, and particularly relates to a noninvasive hemoglobin detector.

### BACKGROUND

Hemoglobin is a kind of protein for transporting oxygen and is composed of globin and heme. Globin and heme may make blood in red color. There is a normal range for an amount of hemoglobin in a human body, usually 120-160 grams (g) per liter (L) for a man and 110-150 g/L for a woman. An increase or a decrease of a hemoglobin concentration often indicates an occurrence of some diseases, such as congenital heart disease, cor pulmonale and anemia caused by various reasons.

A current clinical method for detecting hemoglobin is to draw blood for tests, such as routine blood or blood gas analysis. However, there are obvious severe defects in the current detection method. Firstly, since a waiting duration for obtaining a required hemoglobin result is long, and a judgment of a condition may be delayed, diagnosis and treatment or rescue may be negatively affected subsequently. Secondly, clinical observation shows that in patients with acute massive blood loss or fluid loss caused by various reasons, there are often different degrees of blood concentration. Deviation between detection data of severe patients and true hemoglobin contents may reach 2-3 times, thus greatly affecting judgments of real situations of patients' hemoglobin during medical treatments and delaying treatments. At present, there is still no quick and effective way to solve above problems in the world.

Through long-term clinical practices, the inventor has found out that an influence of blood concentration on a result of a routine blood test may be largely eliminated through a color change of a palpebral conjunctiva of a patient, so a real situation of hemoglobin of the patient may be quickly and efficiently judged, and dilemmas faced by current clinical treatments may be avoided. However, this method needs long-term clinical observation and experience accumulation of medical workers.

### SUMMARY

In order to solve above problems, the disclosure provides a noninvasive hemoglobin detector. The detector selects an image of a palpebral conjunctiva with a closest color by sampling an image of a palpebral conjunctiva of a patient and comparing the image with images of palpebral conjunctivae stored in a database, and finally measures a hemoglobin value of the patient.

In order to achieve above objectives, the disclosure provides a following scheme.

The noninvasive hemoglobin detector includes an image acquisition system, an image processing and analysis system and a printing device.

The image acquisition system is connected with the image processing and analysis system, and the image acquisition system is used for acquiring, transmitting and storing an image to be tested.

The image processing and analysis system is further connected with the printing device, and the image processing and analysis system is used for processing and analyzing the image to be tested to obtain an analysis result.

The printing device is used for printing the image to be tested and the analysis result.

Optionally, the image acquisition system includes an image acquisition module, a first transmission module, a first storage module and a display module.

The image acquisition module is connected with the first transmission module, and the image acquisition module is used for acquiring the image to be tested.

The first transmission module is connected with the image processing and analysis system, and the first transmission module is used for transmitting the image to be tested to the image processing and analysis system.

The first storage module is connected with the image acquisition module, and the first storage module is used for storing the image to be tested.

The display module is connected with the image processing and analysis system, and the display module is used for displaying the analysis result.

Optionally, the image acquisition module includes a miniature camera and a control button.

The miniature camera is used for acquiring the image to be tested.

The control button is used for controlling the miniature camera for image acquisition.

Optionally, the display module includes a touchable display screen and an early warning device, where the touchable display screen is used for displaying the analysis result.

The early warning device is used for issuing an early warning sound based on the analysis result.

The touchable display screen is also used for inputting an age and a gender of a person to be tested.

Optionally, the image processing and analysis system includes an image processing module, a second transmission module, a database module and a second storage module.

The image processing module is connected with the second transmission module, and the image processing module is used for processing the image to be tested to obtain a first image.

The second transmission module is further connected with the database module, and the second transmission module is used for receiving the image to be tested and transmitting the analysis result to the display module.

The database module is further connected with the printing device, and the database module is used for storing contrast images and obtaining the analysis result based on the contrast images.

The second storage module is connected with the database module and the printing device, and the second storage module is used for storing the image to be tested.

Optionally, a method for processing the image to be tested includes: using a principle of image subtraction, subtracting a background, and obtaining an image with a background with a zero R, a zero G and a zero B.

Optionally, the database module includes a contrast database and an analysis module.

The contrast database is connected with the analysis module, and the contrast database is used for storing contrast images with manual calibration information.

The analysis module is connected with the image processing module, and the analysis module is used for obtaining the analysis result based on the first image and the contrast images.

Optionally, the manual calibration information includes ages, genders and hemoglobin concentrations.

The disclosure has beneficial effects as follows.

According to the disclosure, the image of the palpebral conjunctiva with the closest color is selected by sampling the image of the palpebral conjunctiva of the patient and comparing the image with the images of the palpebral conjunctivae stored in the database, and finally the hemoglobin value of the patient is measured. The disclosure has a simple structure, convenient operation, and may obtain a hemoglobin concentration range of the patient in a short time for timely development of rescue plans.

### BRIEF DESCRIPTION OF THE DRAWING

In order to explain technical schemes of the disclosure more clearly, a drawing needed in embodiments is briefly introduced below. Obviously, the drawings in a following description are only some embodiments of the disclosure. For ordinary people skilled in the field, other drawings may be obtained according to these drawings without paying a creative labor.

FIG. 1 is a schematic structural diagram of a noninvasive hemoglobin detector according to the disclosure.

### DETAILED DESCRIPTION OF THE EMBODIMENTS

In the following, technical schemes in embodiments of the disclosure may be clearly and completely described with reference to attached drawings. Obviously, the described embodiments are only a part of the embodiments of the disclosure, but not all embodiments. Based on the embodiments in the disclosure, all other embodiments obtained by ordinary technicians in the field without a creative labor belong to a scope of protection of the disclosure.

In order to make above objects, features and advantages of the disclosure more obvious and easier to understand, the disclosure may be further described in detail with the attached drawings and specific embodiments.

### Embodiment 1

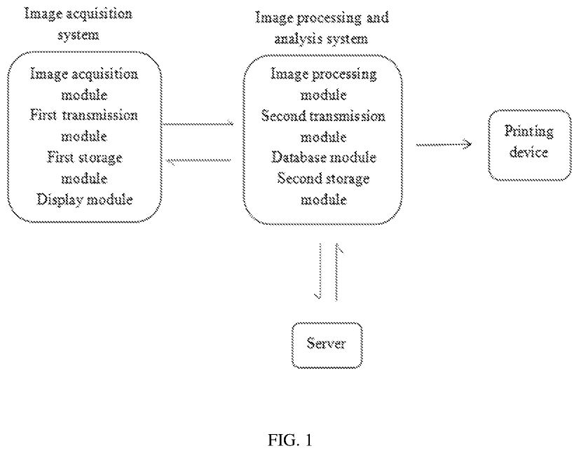

A structural schematic diagram of a noninvasive hemoglobin detector according to the disclosure is shown in FIG. 1, including an image acquisition system, an image processing and analysis system and a printing device.

The image acquisition system is connected with the image processing and analysis system, and includes an image acquisition module, a first transmission module, a first storage module and a display module.

The image acquisition module is connected with the first transmission module, and the image acquisition module includes a miniature camera and a control button. The miniature camera is used to collect an image of a palpebral conjunctiva of a patient. The control button is used to control the miniature camera for image acquisition.

The first transmission module is connected with the image processing and analysis system, and the first transmission module is used for transmitting the image of the palpebral conjunctiva of the patient to the image processing and analysis system.

The first storage module is connected with the image acquisition module, and the first storage module is used for storing the image of the palpebral conjunctiva of the patient.

The display module is connected with the image processing and analysis system, and the display module includes a touchable display screen and an early warning device, where the touchable display screen is used for displaying a hemoglobin concentration range of the patient. The touchable display screen is also used for medical staff to input information such as an age and a gender the patient after collecting the image of the palpebral conjunctiva of the patient. The early warning device is used to send out an early warning sound when the hemoglobin concentration range of the patient is analyzed to exceed or fall below a normal level, so as to remind the medical staff to take rescue measures in time.

The image processing and analysis system is further connected with the printing device, and includes an image processing module, a second transmission module, a database module and a second storage module.

The image processing module is connected with the second transmission module. The image processing module is used for subtracting a background of the image of the palpebral conjunctiva of the patient by using a principle of image subtraction to obtain an image with a background with a zero R, a zero G and a zero B, and performing three-dimensional conversion on the image of the palpebral conjunctiva of the patient from which the background is removed to obtain a three-dimensional vector of the image of the palpebral conjunctiva of the patient.

The database module is connected with the printing device, and includes a contrast database and an analysis module. The contrast database is connected with the analysis module, and the contrast database includes manually calibrated contrast images. A processing method of the images in the contrast database is consistent with that of an image of a palpebral conjunctiva of a patient to be tested. Manual calibration information includes ages, genders and hemoglobin values of persons to whom the contrast image belong. The contrast database is grouped according to the ages and the genders. A maximum limit and a minimum limit are set for each group. When the hemoglobin concentration range of the patient is detected to be higher than the maximum limit or lower than the minimum limit, the display module gives an early warning.

The analysis module is used for comparing and analyzing the contrast images and the image of the palpebral conjunctiva of the patient to obtain an analysis result.

A working process of the analysis module includes:

defining f=(f₁, f₂, f₃) as a color three-dimensional vector of an image of a palpebral conjunctiva of a patient to be tested, and g=(g₁, g₂, g₃) as a color three-dimensional vector of an image in the contrast database; and

calculating a chroma saturation similarity coefficient and a brightness similarity coefficient.

A calculation formula of the chroma saturation similarity coefficient is as follows:

$$r ( f , g ) = \frac{{f}_{1} {g}_{1} + {f}_{2} {g}_{2} + {f}_{3} {g}_{3}}{\sqrt{( {f}_{1}^{2} + {f}_{2}^{2} + {f}_{3}^{2} ) ( {g}_{1}^{2} + {g}_{2}^{2} + {g}_{3}^{2} )}} ,$$

where the greater the value of r(f,g), the higher the similarity.

A calculation formula of the brightness similarity coefficient is as follows:

$$k ( f , g ) = \frac{\vert {f}_{1} + {f}_{2} + {f}_{3} - {g}_{1} - {g}_{2} - {g}_{3} \vert}{765} ,$$

where the greater the value of k(f,g), the smaller the brightness difference.

Color similarities are calculated according to weighting coefficients.

A calculation formula of the color similarities is as follows:

s(f, g)=αr(f, g)+βk(f, g),

where α is a weighting coefficient of the chroma saturation similarity coefficient, and β is a weighting coefficient of the brightness similarity coefficient; and α+β=1 and α>β. In this embodiment, α is taken as 0.7 and β is taken as 0.3.

The color similarities are compared, and a hemoglobin concentration of a image with a highest similarity is taken as a hemoglobin concentration value of image data of this test. A similarity threshold is set as >0.9. When the similarity of the image with the highest similarity is lower than 0.9, an error alarm is sent out and the medical staff is reminded to re-collect an image of the person to be tested.

The second transmission module is used for receiving the image of the palpebral conjunctiva of the patient, transmitting the image to the image processing module for corresponding image processing, and transmitting the analysis result obtained by the analysis module to a data acquisition system.

The second storage module is connected with the printing device, and the second storage module is used for storing the image of the palpebral conjunctiva of the patient and the corresponding analysis result obtained by the analysis module.

The printing device is used for printing the image of the palpebral conjunctiva of the patient and the analysis result.

The second storage module may also be connected with a hospital database and a large printer through a server, so that it is convenient for the patient to retrieve a corresponding image of a palpebral conjunctiva and a corresponding analysis result when printing all examination images and result reports in later periods.

Infancy is taken as an example in this embodiment. A normal hemoglobin concentration range for newborns is 150-210 g/L. If a hemoglobin value is lower than 145 g/L, a newborn is regarded as anemia. A baby aged 1-3 months is in a period of physiological anemia, and a normal hemoglobin concentration range is 90-110 g/L. If a hemoglobin value is lower than 90 g/L, the baby is regarded as anemia. A normal hemoglobin concentration range of a baby aged 4-6 months is 100-110 g/L. If a hemoglobin value is lower than 100 g/L, the baby is regarded as anemia. A normal hemoglobin concentration range of a child aged 0.5-1 year is 110-120 g/L. If a hemoglobin value is lower than 110 g/L, the child is regarded as anemia.

It is worth noting that the hemoglobin value of manually calibrated image data is specific. When information of the image of the palpebral conjunctiva of the patient has the highest similarity with a picture in the contrast database through a comparative analysis, an output hemoglobin range is an interval range of the hemoglobin value. For example, if the image of the palpebral conjunctiva of the patient has the highest similarity with image data with a manually marked hemoglobin value of 105 g/L in the contrast database, the output hemoglobin concentration range is 100-110 g/L. This embodiment takes upper and lower tens of the hemoglobin value as the interval range.

For example, if an age of a person to be tested is 4 months and a gender is female, collected image data is compared with images of girls aged 4-6 months in the contrast database, and picture data with a highest similarity is obtained by an analysis, and a hemoglobin value of the image data is read and a range of the hemoglobin value is output.

### Embodiment 2

This embodiment introduces a working process of a system in detail in combination with each module according to the disclosure.

A contrast database is composed of a large number of images of palpebral conjunctivae of patients with known hemoglobin concentrations, and the images are calibrated manually with ages, genders and hemoglobin concentration values as judgment criteria of the image of the palpebral conjunctiva of the patient patient to be tested. The images are divided into a test set and a training set according to a certain proportion, and the analysis module is trained and tested for an accuracy separately, so as to adjust coefficients of the color similarities and make a measurement accuracy of the noninvasive hemoglobin detector according to the disclosure reach a higher level.

In a use process, the medical staff performs image acquisition of a palpebral conjunctiva of a patient through the control button to obtain the image of the palpebral conjunctiva of the patient, and inputs information of an age and a gender of the patient to the detector through the touchable display screen. After the image is processed by the image processing module, the image is transmitted to the analysis module, and a similarity is compared between the image and a corresponding group in the contrast database based on the age and the gender, and a corresponding image in the contrast database with a highest similarity is selected. A corresponding manually marked hemoglobin concentration of the image is read, and a corresponding interval range of the hemoglobin concentration is determined. The transmission module transmits the interval range to the image acquisition module to display the analysis result. When a similarity value with the highest similarity is less than 0.9, the information is transmitted to the display module and displayed as an error, and the medical staff is reminded to re-collect an image of the palpebral conjunctiva of the patient. When the concentration range obtained from the analysis result is higher than a preset maximum limit or lower limit than a minimum limit, the early warning device sends out an early warning sound to attract an attention of the medical staff to remind the medical staff to take rescue measures in time.

Moreover, the contrast database may store some emergency rescue knowledge empirically, and display corresponding rescue information when the hemoglobin value of the person to be tested is higher or lower than a normal range.

The above-mentioned embodiments are only descriptions of preferred modes of the disclosure, and do not limit a scope of the disclosure. Under a premise of not departing from a design spirit of the disclosure, various modifications and improvements made by ordinary technicians in the field to the technical scheme of the disclosure shall fall within a protection scope determined by claims of the disclosure.

What is claimed is:

1. A noninvasive hemoglobin detector, comprising: an image acquisition system, an image processing and analysis system and a printing device;
the image acquisition system is connected with the image processing and analysis system, and the image acquisition system is used for acquiring, transmitting and storing an image to be tested;
the image processing and analysis system is further connected with the printing device, and the image processing and analysis system is used for processing and analyzing the image to be tested to obtain an analysis result; and
the printing device is used for printing the image to be tested and the analysis result.

2. The noninvasive hemoglobin detector according to claim 1, wherein the image acquisition system comprises an image acquisition module, a first transmission module, a first storage module and a display module;
the image acquisition module is connected with the first transmission module, and the image acquisition module is used for acquiring the image to be tested;
the first transmission module is connected with the image processing and analysis system, and the first transmission module is used for transmitting the image to be tested to the image processing and analysis system;
the first storage module is connected with the image acquisition module, and the first storage module is used for storing the image to be tested; and
the display module is connected with the image processing and analysis system, and the display module is used for displaying the analysis result.

3. The noninvasive hemoglobin detector according to claim 2, wherein the image acquisition module comprises a miniature camera and a control button;
the miniature camera is used for acquiring the image to be tested; and
the control button is used for controlling the miniature camera for image acquisition.

4. The noninvasive hemoglobin detector according to claim 2, wherein the display module comprises a touchable display screen and an early warning device, wherein the touchable display screen is used for displaying the analysis result;
the early warning device is used for issuing an early warning sound based on the analysis result; and
the touchable display screen is also used for inputting an age and a gender of a person to be tested.

5. The noninvasive hemoglobin detector according to claim 1, wherein the image processing and analysis system comprises an image processing module, a second transmission module, a database module and a second storage module;
the image processing module is connected with the second transmission module, and the image processing module is used for processing the image to be tested to obtain a first image;
the second transmission module is further connected with the database module, and the second transmission module is used for receiving the image to be tested and transmitting the analysis result to the display module;
the database module is further connected with the printing device, and the database module is used for storing contrast images and obtaining the analysis result based on the contrast images; and
the second storage module is connected with the database module and the printing device, and the second storage module is used for storing the image to be tested.

6. The noninvasive hemoglobin detector according to claim 5, wherein a method for processing the image to be tested comprises:
using a principle of image subtraction, subtracting a background, and obtaining an image with a background with a zero R, a zero G and a zero B.

7. The noninvasive hemoglobin detector according to claim 5, wherein the database module comprises a contrast database and an analysis module;
the contrast database is connected with the analysis module, and the contrast database is used for storing contrast images with manual calibration information; and
the analysis module is connected with the image processing module, and the analysis module is used for obtaining the analysis result based on the first image and the contrast images.

8. The noninvasive hemoglobin detector according to claim 7, wherein the manual calibration information comprises ages, genders and hemoglobin concentrations.
